(19) United States
(12) Patent Application Publication
(10) Pub. No.: US 2003/0229377 A1
(43) Pub. Date: Dec. 11, 2003

(54) Tube and rod suture clamping system

(76) Inventor: Thomas Tong, Union City, CA (US)

Correspondence Address: T. Gene Dillahunty, BURNS, DOANE, SWECKER & MATHIS, L.L.P., P.O. Box 1404, Alexandria, VA, 22313-1404, US

(21) Appl. No.: 10/164,606

(22) Filed: Jun. 10, 2002

(51) Int. Cl. A61B 17/04

(52) U.S. Cl. 606/232

(57) ABSTRACT

A suture clamping system, including a rod; and a clamp having a first end into which an end of the rod is received and a second end dimensioned to be snap-fit around the rod, the first and second ends being separated by a bendable mid-section. A method of clamping a suture, including positioning the suture between a clamp and a rod, wherein the clamp has a C-shaped cross section and is dimensioned to be snap-fit around the rod; and pushing the clamp against the rod such that the clamp snap-fits around the rod, thereby trapping the suture between the clamp and the rod.

### TECHNICAL FIELD

The present invention relates to systems for securing a pair of suture lengths together or for using a single strand of suture to secure tissues together at an operative site in a patient without necessarily tying a knot.

### BACKGROUND OF THE INVENTION

Sutures are used to sew tissue together, and thereby close tissue openings, cuts or incisions during or after any of a very wide variety of medical procedures. Typically, the surgeon manually ties together a suture pair to close the opening, however, automatic suture tying systems have also been developed.

There are a number of disadvantages of knotting sutures together to secure tissues to one another. For example, manual knot tying requires considerable dexterity. Also, manual knot tying can take considerable time. Knot tying is further complicated by the fact that surgical sutures have low friction surfaces. Therefore, it is typically necessary for a surgeon to include many “throws” when tying the knot. This multiple-throw problem occurs even if an automatic knot tying device is used. Unfortunately, as the number of loops or “throws” incorporated into the knot increase, the knot becomes increasingly large and bulky. Moreover, the surgeon typically needs to handle strands of adequate suture length prior to commencing manual knot tying. Thus, manual knot tying requires considerable space both in which to view, and to perform, the actual suture knot tying. Therefore, knot tying is particularly difficult in areas of limited available space or access, for example, at the back of the patient's heart during a coronary artery bypass graft (CABG) operation. Manually tied knots often lock prior to reaching the intended amount of tension to be applied to the tissue. Furthermore, tissues are typically secured together by a pair of sutures wherein each of the sutures in the pair pass through both of the tissues which are secured together by tying off the suture pair. It would instead be advantageous to provide a system which is adapted to secure together two tissues, but uses only a single strand of suture.

### SUMMARY OF THE INVENTION

The present invention provides a suture clamping system, including a rod and a clamp. The clamp preferably has a first end into which an end of the rod is received and a second end which is dimensioned to be snap-fit around the rod. Preferably, the first and second ends of the clamp are separated by a bendable mid-section. In various embodiments, a notch is formed in the second end of the clamp for receiving one or more sutures (e.g.: a suture pair) therebetween.

In accordance with a preferred method, the one or more sutures are clamped by first positioning the suture(s) between a clamp and a rod, wherein the clamp has a C-shaped cross section and is dimensioned to be snap-fit around the rod; and then pushing the clamp against the rod such that the clamp snap-fits around the rod, thereby trapping the suture(s) between the clamp and the rod. In preferred aspects, a suture pair is clamped together, however, in alternate preferred aspects, the clamp and rod are simply secured to a single suture strand. In those preferred aspects of the invention in which a pair of sutures are used, the suture pair is trapped between the rod and the clamp as the clamp is snap-fit around the rod.

In various aspects, the rod is tubular in shape, and the clamp is dimensioned to be snap or press fit around a portion of the circumference of the rod. In optional preferred aspects, the portion of the circumference of the rod encircled by the C-section of the clamp is greater than 180 degrees such that the C-section of the clamp “latches” firmly around the rod. In various exemplary embodiments the portion of the circumference of the rod encircled by the C-section of the clamp is greater than 200 degrees, or more preferably 220 degrees, or more preferably 230 degrees or more preferably 240 degrees.

The present system may further include an applicator which positions the clamping system, and which forces the clamping system into a closed position in which the clamp is snap-fit around the rod. Optionally, the applicator may comprise a pair of pincers which are dimensioned to hold the rod and clamp therebetween.

The present invention may be used so as to completely avoid manual suture knot tying. Therefore, it also avoids the bulky multiple loops or “throws” required when knot tying. Instead, with the present system, the suture(s) is simply “clamped” together between the rod and the clamp. Thus, the present invention saves valuable time, and can be operated in very small spaces with limited surgical access, and does not require the long lengths of suture which are otherwise needed for manual knot tying.

The present clamping system can be positioned directly adjacent to the operative site at which it is desirable to secure the suture(s). Thus, the present applicator can be used to fasten together a suture pair right at the operative site. Therefore, the present system may be used so that it does not pull suture at the surgical site when the suture(s) are clamped or secured together. Rather, as the present system is deployed, it simply closes to lock together the suture(s) at the operative site. Therefore, the present system can easily be positioned right up against the operative site. In contrast, knot tying may either result in a loose knot being positioned at a small distance away from the operative site, or an overly tight knot pulling excessively on the tissues.

Thus, the present invention provides a rapid, easy to operate, system to secure a suture pair together, or to clasp onto a single strand of suture.

### BRIEF DESCRIPTION OF THE DRAWINGS

FIG. 1A is a side elevation view of a first aspect of the present invention in an open position.

FIG. 1B is an exploded view corresponding to FIG. 1A.

FIG. 2 is a side elevation view of a first aspect of the present invention in a closed position.

FIG. 3A is a front elevation view of the present invention in an open position.

FIG. 3B is an exploded view corresponding to FIG. 3A.

FIG. 4 is a front elevation view of the present invention in a closed position.

FIG. 5 is a perspective view of the distal end of an applicator adapted to hold the system of FIGS. 1 to 4.

FIG. 6 is a side elevation view corresponding to FIG. 5, with the rod and clamp in an open position.

FIG. 7 is a side elevation view corresponding to FIG. 5, with the rod and clamp in a closed position.

FIG. 8 is a perspective view of a second embodiment of the present invention.

FIG. 9 is a top plan view of the embodiment of FIG. 8.

FIG. 10 is a perspective view of a third embodiment of the present invention.

FIG. 11 is a perspective view of the present system securing tissues together using only a single strand of suture.

### DETAILED DESCRIPTION OF THE DRAWINGS

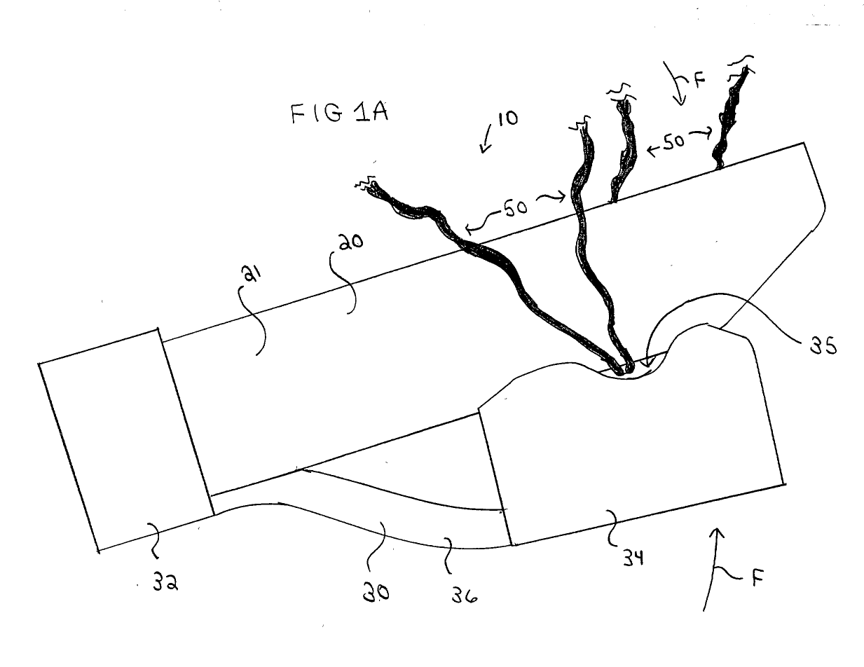

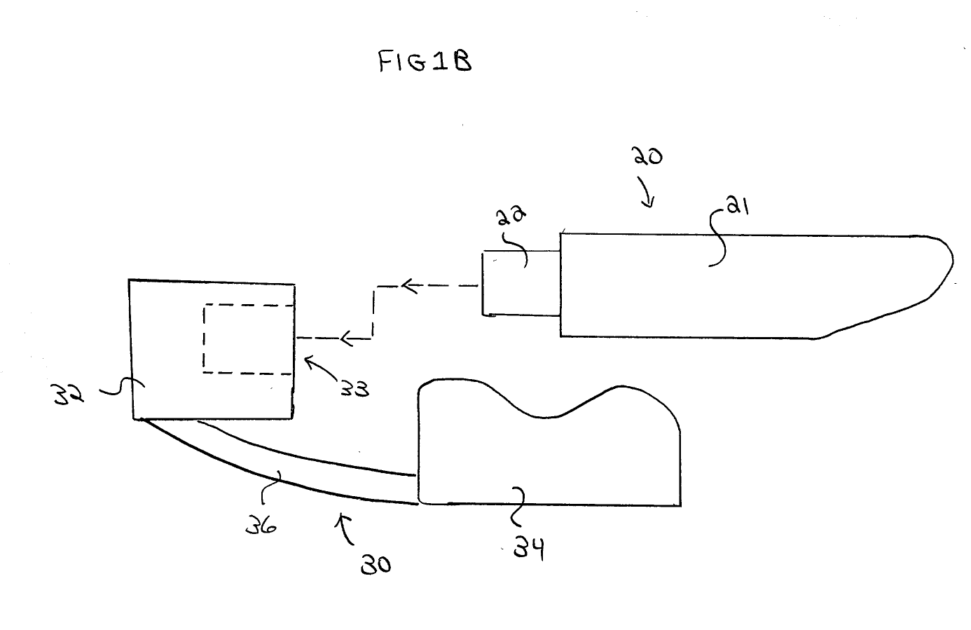

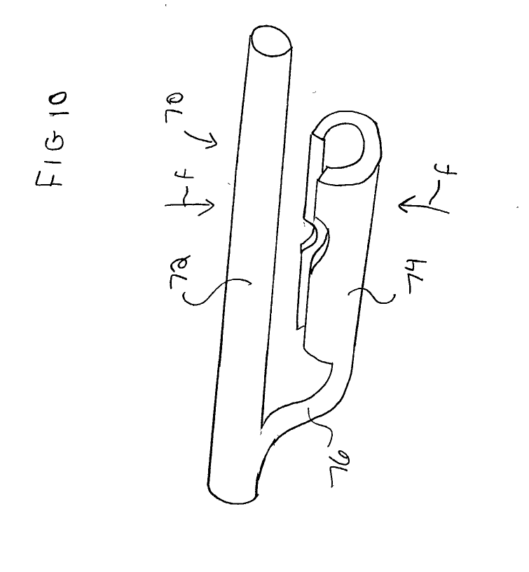

The present invention provides a suture clamping system which is fast and easy to use, and avoids the need for knot tying. FIGS. 1 to 7 show a first (two-part) embodiment of the invention comprising a separate rod and clamp. FIGS. 8 and 9 show a second (one-part) embodiment of the invention comprising a structure folded back on itself. FIG. 9 shows a third embodiment of the invention comprising a fused rod and clamp portion. The various embodiments of the invention provide a structure that clamps a length of suture or a suture pair between two portions of the structure. The portions of the structure generally include a tube and a rod.

Referring first to FIGS. 1A and 1B, a two part suture clamping assembly 10 is provided. Clamping assembly 10 includes a rod 20 and a clamp 30. Clamp 30 has a first end 32, a second end 34 and a bendable mid-section 36. Preferably, a notch 35 is provided in second end 34. As shown in FIG. 1B, rod 20 has a narrow end 22 which is dimensioned to be received into bore 33 in end 32 of clamp 30.

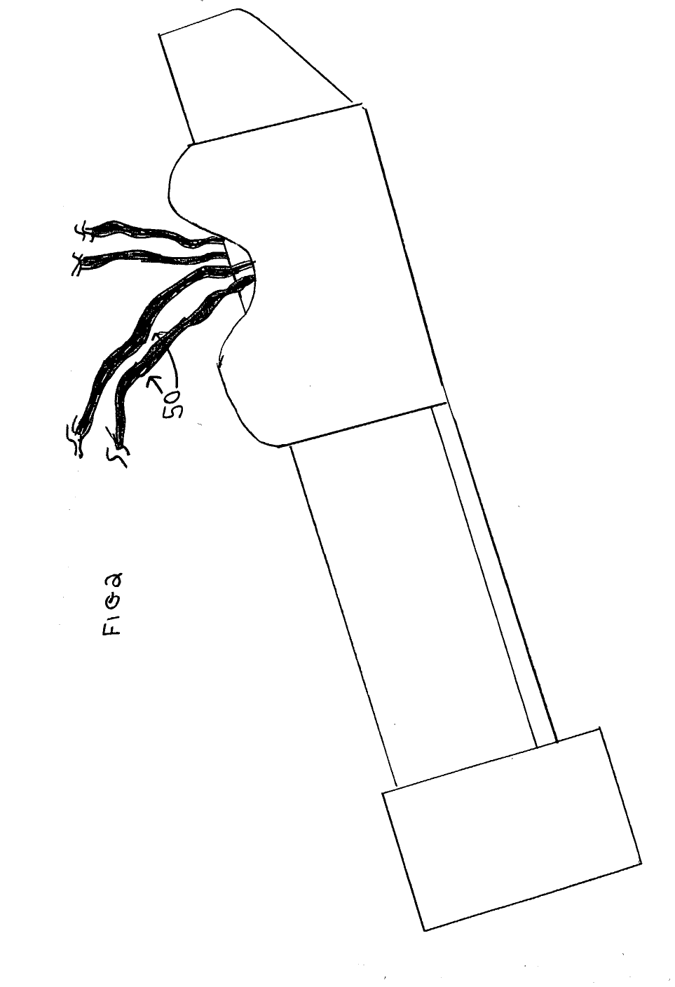

In accordance with a preferred method of operation, one or more sutures 50 (e.g.: a suture pair) is preferably received into notch 35, such that it passes between rod 20 and clamp 30. Thereafter, clamping assembly 10 is “closed” (i.e. “deployed”) with end 34 of clamp 30 being snap-fit around rod 20. Thus, suture pair 50 is clamped together as the suture pair is effectively trapped between rod 20 and end 34 of clamp 30. Specifically, to move assembly 10 from an “open” position (FIG. 1A) to a “closed” position (FIG. 2), a pinching force F is applied, which simply pinches together rod 20 and end 34 of clamp 30.

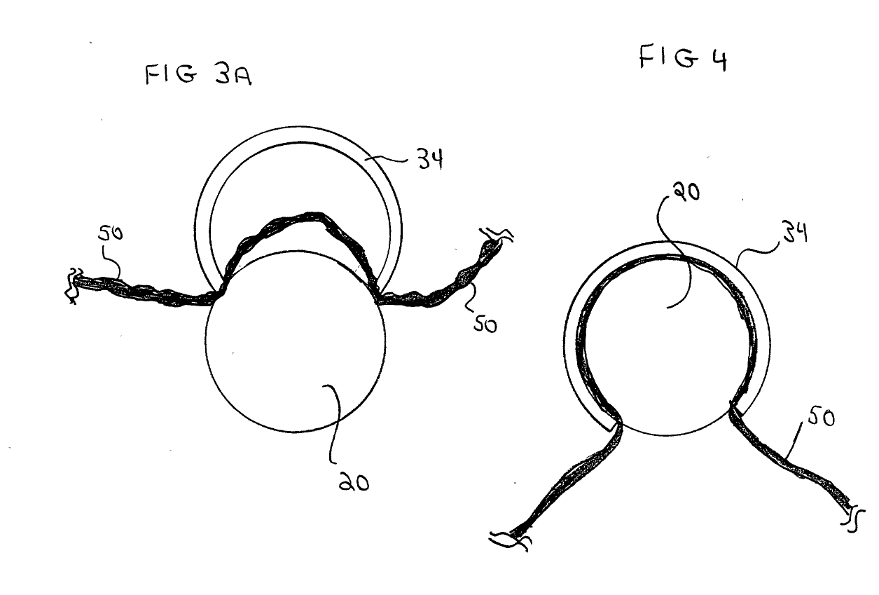

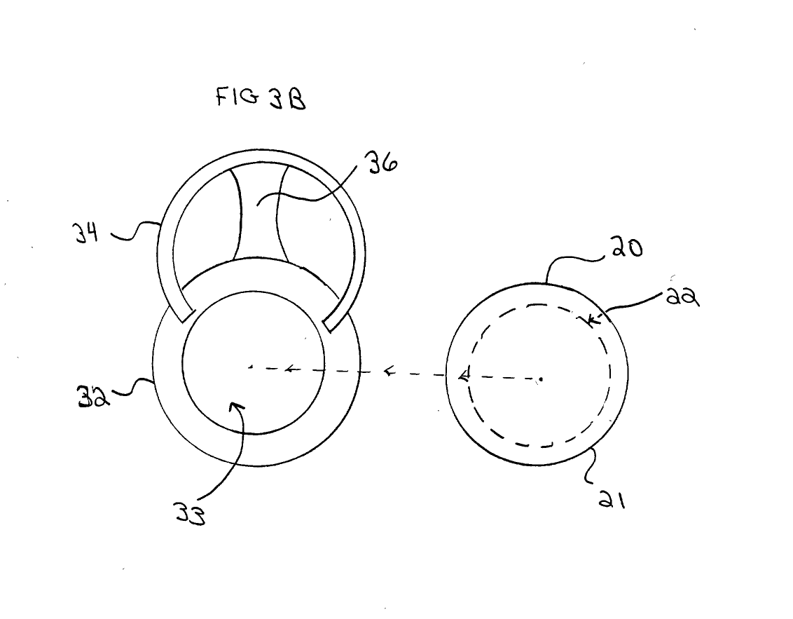

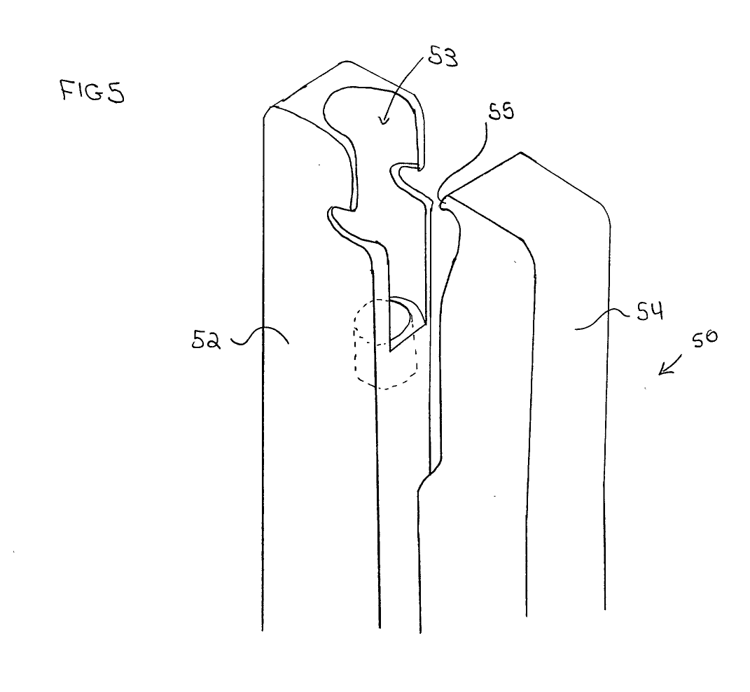

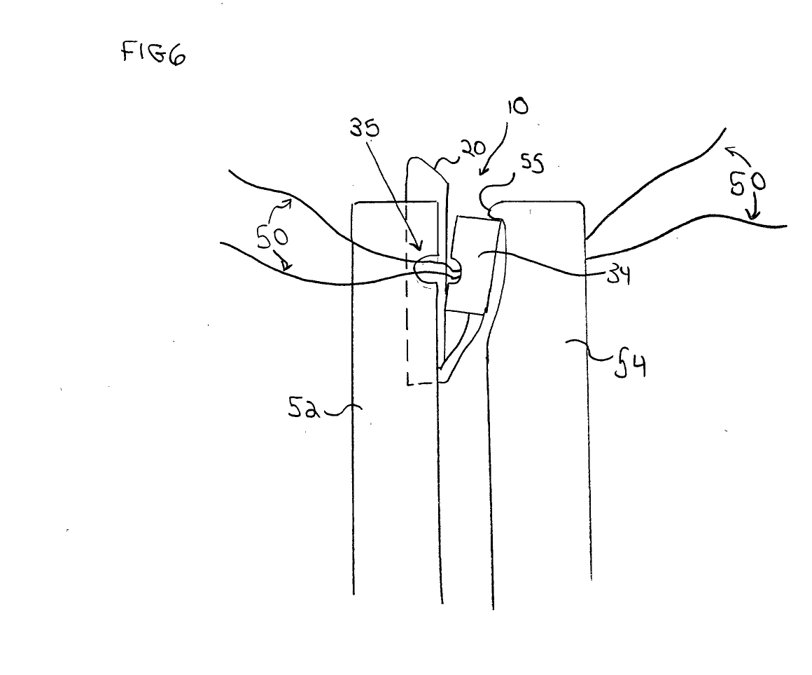

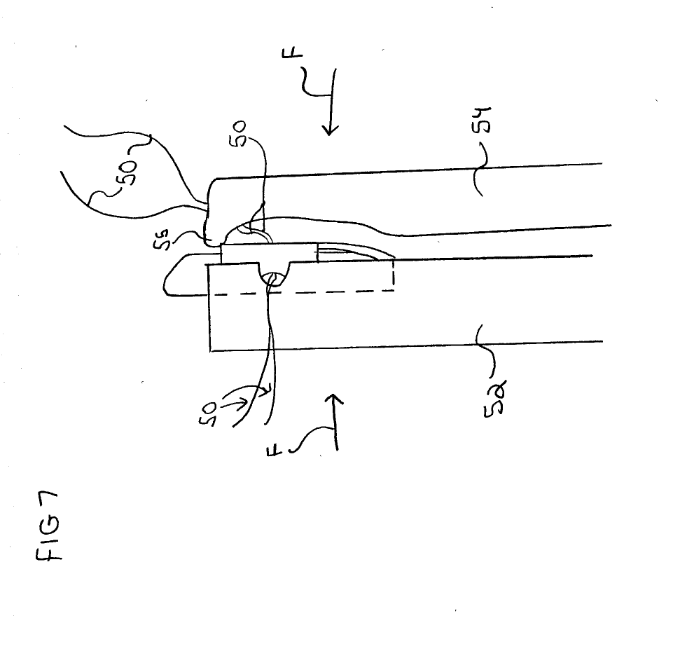

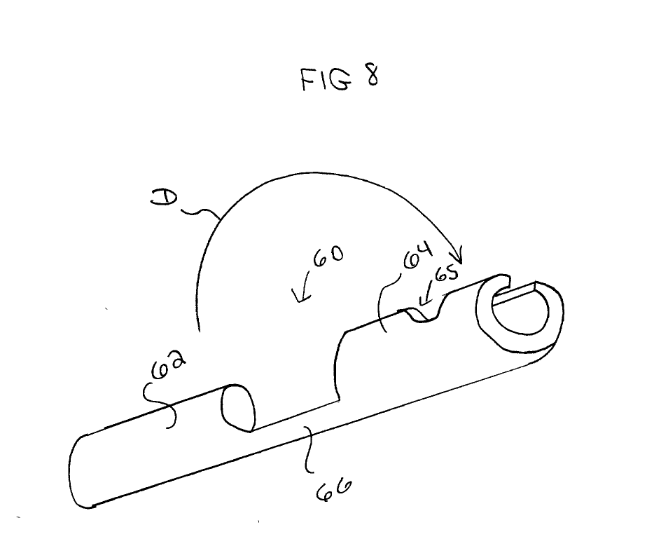

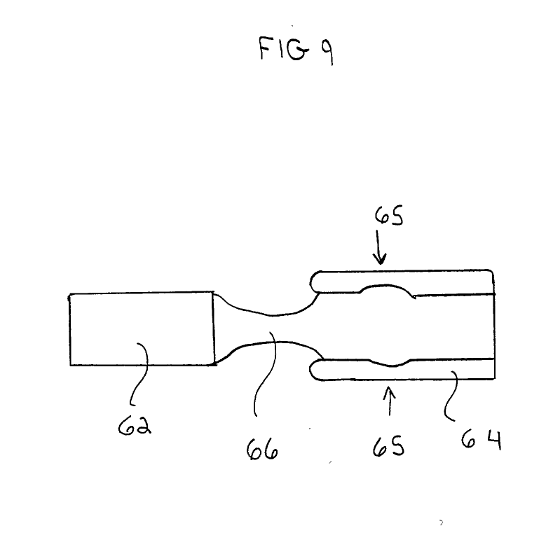

As can be seen in FIGS. 3A to 4, end 34 has a C-shaped cross section, which is snap-fit around rod 20. Rod 20 and clamp 30 are each preferably dimensioned such that an interference fit is created therebetween which compresses suture 50 between rod 20 and clamp 30. The compressive force aids in holding the suture in place. Alternatively, rod and clamp may be dimensioned to be line-to-line fit and the suture material provides the interference. Another alternative is that the rod and clamp may be dimensioned to have a small space or gap that is slightly smaller than the intended suture size to have adequate compression.

In optional aspects, rod 20 and clamp 30 are made of biocompatible steel, plastic or a shape memory metal such as Nitinol™. Rod 20 may optionally have a roughened surface 21 (FIG. 1B) to increase the sliding friction against suture pair 50. Surface 21 may be roughened by a variety of techniques, including sandblasting, filing, knurling and chemical etching.

Preferably, assembly 10 is sized approximately for the intended suture size. Preferably, assembly 10 has an outer diameter of approximately 0.032″ to secure 7-0 polypropylene sutures. Preferably, assembly 10 has a length of not more than 0.12″ and most preferably, not more than about 0.18″. When the assembly is embodied in such a small size, it can be positioned directly adjacent to the surgical operative site. Alternatively, the assembly may be sized for use with suture in the range of 8-0 to 3-0. In addition, the material of the suture may be polypropylene monofilamenrt, braided Dacron/nylon or similar suture.

Due to its very small size, in accordance with the present invention, an optional applicator is provided. Referring to FIGS. 5 to 7, applicator 50 comprises a “pinching” device having prongs 52 and 54. As shown in Figs. prong 52 preferably has a recess 53 into which end 32 of assembly 10 is received. Prong 54 preferably has a stub (e.g.: an overhang) 55 at its distal end.

As shown in FIG. 6, assembly 10 is first held in its open position between prong 52 and 54. At this time, a suture pair 50 is positioned in notch 35 between end 34 of clamp 30 and rod 20. As is shown in FIG. 7, a force F is then applied, pinching together prongs 52 and 54, thereby pushing clamp end 34 into a snap-fit position over rod 20. Thus, suture pair 50 is clamped into position between clamp end 43 and rod 20. Thereafter, prongs 52 and 54 can again be opened (to the position shown in FIG. 6). At this time, assembly 10 remain firmly clamped around suture pair 50. Thus, assembly 10 can easily be removed from recess 53 of applier 50.

A second embodiment of the present invention is shown in FIGS. 8 and 9. This embodiment is similar in function to the first embodiment, however, it is instead formed from a single block of material, as follows. Clamp 60 is formed with a tubular end 62, an opposite end 64 (having a C-shaped cross-section) and a thin bendable mid-section 66. Alternatively, the different sections of clamp 60 can be separate pieces which are joined together. In this case, the separate pieces can be made of different materials. In accordance with this embodiment of the present invention, clamp 60 is folded over upon itself (folding end 62 in direction D) such that end 62 is snap-fit into end 64. Thus, a suture pair (not shown) can be held together between ends 62 and 64 (similar to the manner in which suture pair 50 is held between rod 20 and clamp 30 in the first embodiment). Preferably, a notch 65 is cut into end 64 such that a single suture or suture pair (not shown) can more easily be positioned therein prior to end 62 being snap-fit into end 64.

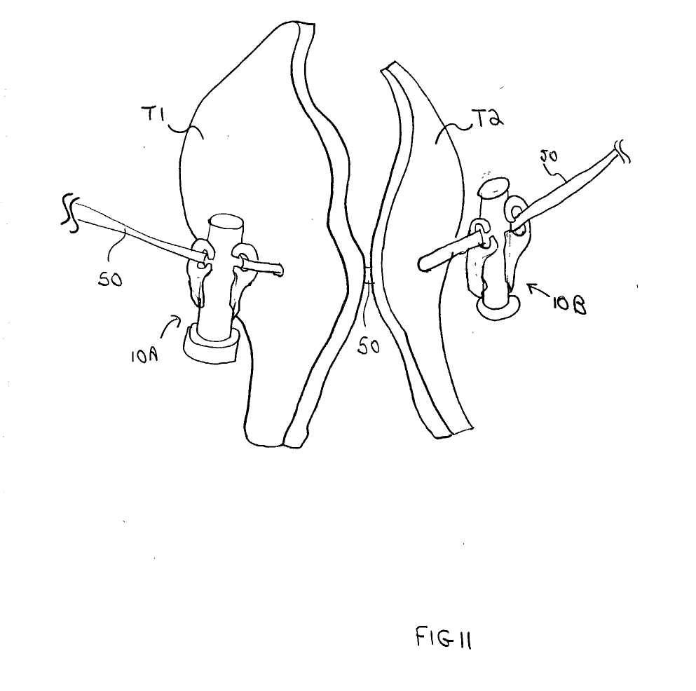

An optional third embodiment is shown in FIG. 10 in which clamping assembly 70 is provided. Assembly 70 comprises a rod portion 72, a C-shaped cross-sectional clamp portion 74 and a bendable member 76 separating them. In preferred aspects, assembly 70 is formed from a single block of material, but it may also be formed from two blocks of material fused or adhesively held together. Assembly 70 operates similar to the first and second embodiments of the invention. Specifically, when a pinching force F is applied, clamp portion 74 is snap-fit into position around rod portion 72 such that a suture pair (not shown) can be trapped therebetween.

FIG. 11 illustrates using the present system to secure two tissues together using only a single strand of suture, as follows. A single suture 50 is threaded through tissues T1 and T2. A first clamping assembly 10A is clamped onto suture 50 right next to tissue T1 and a second clamping assembly 10B is clamped onto suture 50 right next to tissue T2. Thus, tissues T1 and T2 are secured together using only a single strand of suture 50. In other aspects of the present invention, (e.g.: as illustrated in FIGS. 1A, 6 and 7) a single clamping assembly 10 may be used to secure a pair of sutures 50 together. Thus, it is to be understood that the present system may be used to clamp around a single suture or a suture pair. Moreover, in the case of a suture pair, such suture pair may comprises two separate strands of suture or opposite ends of a single strand of suture, as desired.

1. A suture clamp system, comprising:
a rod; and
a clamp having a first end connected to an end of the rod, the clamp comprising a second end dimensioned to be snap-fit around the rod, the clamp having a bendable mid-section separated by first and second opposite ends.

2. The system of claim 1, wherein the rod is tubular shaped and the clamp fits around a portion of the circumference of the rod.

3. The system of claim 1, wherein the rod and clamp are dimensioned such that a length of suture may be trapped therebetween.

4. The system of claim 1, further comprising a notch in the second end of the clamp.

5. The system of claim 4, wherein the notch is cut across opposite sides of the second end of the clamp.

6. The system of claim 1, wherein the rod is tapered towards the second end of the clamp.

7. The system of claim 1, wherein the outer surface of the rod is roughened.

8. The system of claim 1, wherein the rod is formed of steel, plastic or a shape memory metal.

9. The system of claim 1, wherein the clamp is formed of steel, plastic or Nitinol™.

10. The system of claim 1, wherein the rod is a hollow tube.

11. The system of claim 1, further comprising:
an applicator which positions the clamping system, and which forces the clamping system into a closed position in which the clamp is snap-fit around the rod.

12. The system of claim 11, wherein the applicator comprises:
a pair of pincers dimensioned to hold the rod and clamp therebetween, the pair of pincers being dimensioned to securely hold the clamping system when the clamping system is in an open position, and to release the clamping system when the clamping system is in a closed position.

13. The system of claim 11, wherein the first pincer comprises a recess dimensioned to receive the first end of the clamp therein and a notch dimensioned to receive a suture pair therethrough, and the second pincer comprises a stub dimensioned to pass over the top of the second end of the clamp.

14. A suture clamping device, comprising:
a first end which is tubular shaped;
a second end which has a C-shaped cross section, the second end being dimensioned to be snap-fit around the first end; and
a bendable mid-section extending between the first and second ends.

15. The system of claim 14, further comprising a notch in the second end.

16. The system of claim 14, wherein the rod and clamp are dimensioned such that a length of suture may be trapped therebetween.

17. A suture clamping device, comprising:
a rod; and
a clamp mounted to the rod, the clamp comprising a C-shaped section and a bendable section, wherein the bendable section is mounted to the rod, and wherein the C-shaped section of the clamp is dimensioned to be snap-fit around the rod.

18. A method of clamping at least one suture, comprising:
positioning the at least one suture between a clamp and a rod, wherein the clamp has a C-shaped cross section and is dimensioned to be snap-fit around the rod; and
pushing the clamp against the rod such that the clamp snap-fits around the rod, the at least one suture being trapped between the clamp and the rod.

19. The method of claim 18, wherein the second end of the clamp comprises a notch.

20. The method of claim 18, wherein the rod and clamp are dimensioned such that a length of suture may be trapped therebetween.

21. The method of claim 18, wherein the clamp has a first end into which an end of the rod is received and a second end which has the C-shaped cross section dimensioned to be snap-fit around the rod.

22. The method of claim 18, wherein the clamp is connected to the rod, and wherein the clamp comprises a bendable mid-section.

23. The method of claim 18, wherein pushing the clamp against the rod comprises:
squeezing the clamp and rod together with a pair of pincers.

24. The method of claim 18, wherein the clamp comprises a notch adjacent to the rod, further comprising:
positioning the at least one suture into the notch prior to pushing the clamp against the rod.

25. The method of claim 18, wherein the at least one suture comprises a suture pair.

26. The method of claim 25, wherein the suture pair comprises opposite ends of a continuous length of suture.
